(19) United States
(12) Patent Application Publication
(10) Pub. No.: US 2016/0235959 A1
(43) Pub. Date: Aug. 18, 2016

(54) DEVICE FOR PUNCTURING HEPARIN CAP AND VASCULAR ACCESS SYSTEM

(71) Applicant: Chengping Hu, Guangzhou (CN)

(72) Inventor: Chengping Hu, Guangzhou (CN)

(73) Assignee: IMPROVE MEDICAL INSTRUMENTS (HUNAN) CO., LTD., Hunan (CN)

(21) Appl. No.: 14/902,295

(22) PCT Filed: Aug. 29, 2013
(86) PCT No.: PCT/CN2013/082549

§ 371 Date: Dec. 30, 2015

(51) Int. Cl. A61M 39/02; A61J 1/20

(52) U.S. Cl. CPC A61M 39/0247; A61J 1/201; A61M 2039/0205; A61M 2039/0258; A61M 2039/027; A61M 2039/0288

(57) ABSTRACT

A device for puncturing a heparin cap is disclosed, including: a connecting unit, adapted to be coupled to a medicinal fluid supply device in fluid communication; a guiding unit, adapted to accommodate at least one part of the heparin cap; and a puncturing unit for puncturing the heparin cap, configured to be in fluid communication with the connecting unit and at least partially located on a predetermined position in the guiding unit. The predetermined position is configured so that in the process of at least one part of the heparin cap entering into the guiding unit, the puncturing unit is inserted into a sealing rubber of the heparin cap at an appropriate location on the heparin cap and an appropriate angle relative to the heparin cap, and ultimately penetrate through the sealing rubber. With the device, the medical worker can complete the puncture of the heparin cap safely, easily and effectively. Meanwhile, the device can greatly reduce or eliminate the unnecessary waste of the medicinal liquid during the subsequent infusion, and facilitates the medical worker to completely wash the medicinal liquid remained within the heparin cap off.

### FIELD

The present disclosure relates to the field of vascular access, and more particularly, to an infusion device for puncturing a heparin cap in a vascular access.

### BACKGROUND

As shown in FIG. 1, a heparin cap generally includes two members: a sealing member 110 and a case body 120. The sealing member is generally made of rubber (such as silicone) or other elastic materials. The case body is generally made of a transparent thermoplastic material with a fluid passage 130 inside the case body, one end of which has a standard 6% (Luer) tapered locking joint 121 adapted to prevent the fixed joint from a falling off accident caused by an unsuitable connection in use for a long-term duration, to maintain the reliability of the connection. Sealing rubber can be embedded through physical pressure into or chemically adhered on the inner wall 122 of one end of the plastic case body away from the fixed joint to form a firmly closed assembly, thereby forming a complete heparin cap 100.

The sealing rubber 110 of the heparin cap allows the pointy needle bar to be inserted into or pulled out of the sealing rubber 110. When the locking joint 121 on the other end of the heparin cap is connected with another device, the inserted needle can establish fluid communication with the device through the fluid passage 130 within the heparin cap. And once the needle is pulled out of the sealing rubber, the sealing rubber can seal itself instantaneously to cut the device off from communication with the outside world. The heparin cap can be frequently or repeatedly punctured.

The heparin cap is often used in cooperation with a long-term used vascular access device, and the medicinal liquid can be infused from outside into a blood vessel through the cooperation between the heparin cap and the vascular access device. The vascular access device may include peripheral short or medium length catheter (such as indwelling needle (PVC)), cardiovascular access device (such as central venous catheter (CVC) and peripherally inserted central catheter (PICC)), and artery catheter. It should be noted that it is also possible to use other types of vascular access devices, including any vascular access device now known or later developed provided it can be used in combination with heparin cap. The specific type of the vascular access device should not constitute to be a limitation for the embodiment described below and the scope of the application.

When the vascular access device works at a non-infusion state, appropriate saline and/or heparin solution can injected into the vascular access device through the heparin cap, to wash or close the vascular access device to prevent formation blood clot formation caused by the medicinal liquid remaining in the vascular access device and blood return.

In addition to the medicinal fluid supply device (such as syringe and infusion apparatus), the device used together with the vascular access device, such as PVC, CVC and PICC, the head of which should be closed by the heparin cap, usually includes an intravenous infusion needle when the medicinal liquid from the vascular access device is infused into the human blood vessel for treatment. FIG. 2 shows a connection mode when these device used together. Firstly, the outer tapered (locking) joint 211 of the medicinal fluid supply device such as syringe 200 is connected to the inner tapered joint 311 of the intravenous infusion needle 300; secondly, the tip 341 of the needle bar of the intravenous infusion needle is inserted into the heparin cap 410 of the vascular access device such as indwelling needle 400 connected to the human blood vessel; finally, the medicinal liquid in the medicinal fluid supply device is infused into the human blood vessel through a fluid passage connecting the medicinal fluid supply device, the intravenous infusion needle and the vascular access device. When the liquid infusion is complete, appropriate amount of heparin solution should be infused to close the vascular access device before pulling the intravenous infusion needle out of the heparin cap, in order to prevent formation blood clot formation caused by the medicinal liquid remaining in the vascular access device and blood return. For the next infusion, the vascular access device should generally be washed by appropriate amount of saline before the infusion operations of the medicinal liquid.

When using the intravenous infusion needle, the operator (such as nurse) needs to remove the protective cover 350 from the needle bar 340 firstly. At this time, the exposed, sharp tip 341 may bring about unexpected injury to the medical worker and/or the patient, and the risk of accident of injury will grow in the subsequent operations. When the steel needle (i.e., needle bar) is puncturing the heparin cap, the medical worker generally holds the heparin cap with the thumb and the index finger of one hand, holds the needle 330 of the infusion needle with the thumb and the index finger of the other hand, and performs cautiously the action of puncture. If the medical worker has a lack of skills, loses focus or operates in wrong way, the following conditions may occur in the puncture course.

(1) The medical worker or the patient may be stabbed by the tip. Since the heparin cap generally has a small shape, and is generally fixed on the skin surface of the patient, the tip is easy to puncture and injure the finger of the medical worker holding the heparin cap and/or the skin of the patient.

(2) The tip may stab on the surface of the plastic inner wall of the heparin cap to produce plastic scraps which may enter the body with the medicinal liquid in the subsequent infusion process; and when the direction in which the tip is inserted is inappropriate, or when the place where the tip is inserted is not deep enough, the tip may be inserted into the plastic inner wall and buried under the plastic body, so the fluid passage cannot to be build between the steel needle and the heparin cap and the subsequent infusion cannot be carried out. The above two conditions may cause a medical accident or bring a potential health risk. Only when the tip is vertically inserted at the central position of the sealing rubber of the heparin, the above results can be effectively avoided.

(3) When washing the vascular access device, the heparin cap is not cleaned completely. In general, the needle bar of the infusion needle is long, and the heparin cap is short, so it is easy to cause the needle bar to be inserted into the heparin cap deeply if uncontrolled or not properly controlled, and when the vascular access device is washed with the saline, the inner wall of the heparin cap close to the sealing rubber is difficult to be washed, and the medicinal liquid is easy to be remained in this place after the vascular access device has been washed. There is likely to cause a medical accident, if there is incompatibility between the medicinal liquid to be infused and the medicinal liquid remained. In addition, the blood products and nutrient solution may provide nutrition for biofilm growth after being remained, so the blood products and nutrient solution should be washed out of the cavity of the catheter.

An intravenous infusion needle has many components, complicated in structure and difficult to manufacture. The unsuitable connection between the needle bar 340, the needle handle 330, the flexible tube 320 and the needle holder (inner tapered joint) 310 may bring insufficient flow, block or leakage to the fluid passage within the whole infusion needle, and may bring accidents to the subsequent medical use. The flexible tube 320 is long (generally not less than 250 mm), so the infusion needle may waste a certain amount of medicinal liquid in use.

The vascular access device is a long-term used medical device which may be kept on the body for a long time. During the usage of the one vascular access device from start to end, the intravenous infusion needle may be used for many times, and the heparin cap may be frequently or repeatedly punctured. As a result, all the risks mentioned above may increase, and the amount of the medicinal liquid wasted may increase.

Based on above, it is required to provide a simple, safe and easily manufactured infusion needle, which allows the medical worker to complete the puncture of the heparin cap safely, easily and effectively without being skilled in the operations and maintenances of PVC, CVC, PICC, three-way valve (devices or ports which require a heparin cap to close its head), and without careful and cautious operations. Meanwhile, the needle can greatly reduce or eliminate the unnecessary waste of the medicinal liquid during the subsequent infusion, and facilitate the medical worker to completely wash off the medicinal liquid remained within the heparin cap.

### SUMMARY

The present disclosure relates to a device and method for puncturing heparin cap and a vascular access system for puncturing heparin cap.

According to a first aspect of the disclosure, a device for puncturing a heparin cap is provided, including: a connecting unit, adapted to be coupled to a medicinal fluid supply device in fluid communication; a guiding unit, adapted to accommodate at least one part of the heparin cap; and a puncturing unit for puncturing the heparin cap, configured to be in fluid communication with the connecting unit and at least partially located on a predetermined position in the guiding unit. The predetermined position is configured so that in the process of at least one part of the heparin cap entering into the guiding unit, the puncturing unit is inserted into a sealing rubber of the heparin cap in an appropriate location in the heparin cap at an appropriate angle relative to the heparin cap, and ultimately penetrate through the sealing rubber.

Preferably, the appropriate angle includes that the puncturing unit is perpendicular to the sealing rubber, and the appropriate location includes a central portion of the sealing rubber.

Preferably, the guiding device also includes a position-limiting component, configured to limit a final depth of the heparin cap entering into the guiding unit. Further, the position-limiting component includes at least one of a bulge arranged within the guiding unit, and a tapered shape of the guiding unit in a direction from the heparin cap to the guiding unit. Further, the final depth can assure the final position of the puncturing unit inserted into the heparin cap to be close to the sealing rubber.

Preferably, at least two of the connecting unit, the guiding unit and the puncturing unit are integrally formed. Further, all of the connecting unit, the guiding unit and the puncturing unit are integrally formed. Further, all of the connecting unit, guiding unit and the puncturing unit are made of a plastic material.

Preferably, at least two of the connecting unit, the guiding unit and the puncturing unit are separately formed, and the detachably assembled. Further all of the connecting unit, the guiding unit and the puncturing unit are separately formed, the outer surface of the puncturing unit is adhered onto the inner surface of the connecting unit, and the connecting unit is fitted into the guiding unit. Further, the connecting unit and the guiding unit are made of a transparent thermoplastic plastic material, and the puncturing unit is made of stainless steel.

Preferably, the coupling between the connecting unit and the medicinal fluid supply device is detachable.

Preferably, the puncturing unit is a needle-shaped component having a fluid passage inside. Further, the puncturing unit is a hollow needle-shaped component.

According to a second aspect of the disclosure, a method for puncturing heparin cap is provided, including holing a device for puncturing a heparin cap according to the first aspect of the disclosure (which may include one or more features in the various above preferred and further features) and a heparin cap respectively; and making the device and the heparin cap approach each other so that in the process of at least one part of the heparin cap entering into the guiding unit, the puncturing unit is inserted into a sealing rubber of the heparin cap in an appropriate location in the heparin cap and at an appropriate angle relative to the heparin cap, and ultimately penetrate through the sealing rubber.

According to a third aspect of the disclosure, a vascular access system is provided, including: a vascular access device; a heparin cap coupled to the vascular access device; a device for puncturing heparin cap according to the first aspect of the disclosure (which may include one or more features in the various above preferred and further features); and a medicinal fluid supply device in fluid communication with the device for supplying medicinal fluid. Preferably, the vascular access device includes at least one of an indwelling needle, a central venous catheter, a peripherally inserted central catheter and an artery catheter.

The present disclosure provides a simple, safe and easily manufactured infusion needle (i.e., a device for puncturing heparin cap), which allows the medical worker to complete the puncture of the heparin cap safely, easily and effectively without being skilled and careful and cautious operations. Meanwhile, the needle can greatly reduce or eliminate the unnecessary waste of the medicinal liquid during the subsequent infusion, and facilitate the medical worker to completely wash off the medicinal liquid remained within the heparin cap.

### BRIEF DESCRIPTION OF THE DRAWINGS

Features, advantages, and technical significance of exemplary embodiments of the disclosure will be described below with reference to the accompanying drawings, in which like numerals denote like elements, and wherein:

FIG. 1 is a cross-sectional view showing a typical heparin cap in the prior art.

FIG. 2 is an exploded cross-sectional view showing a syringe, an intravenous infusion needle and an indwelling needle used together in the prior art.

FIG. 3 is a perspective cross-sectional showing a heparin cap puncture infusion needle according to one embodiment of the disclosure.

FIG. 4 is a cross-sectional view showing the heparin cap puncture infusion needle of FIG. 3.

FIG. 5 is an exploded cross-sectional view showing a partial section of a syringe, the heparin cap puncture infusion needle of FIG. 3, and an indwelling needle used together.

FIG. 6 is a cross-sectional view showing the heparin cap puncture infusion needle of FIG. 3 puncturing the heparin cap.

FIG. 7 and FIG. 8 are cross-sectional views showing heparin cap puncture infusion needles according to two alternative embodiments of the disclosure respectively.

FIG. 9 and FIG. 10 are cross-sectional views showing heparin cap puncture infusion needles according to another two alternative embodiments of the disclosure respectively.

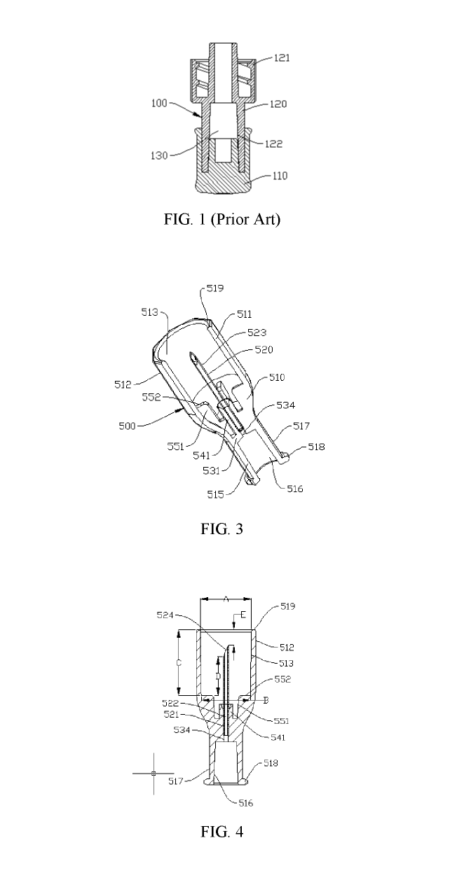

### DETAILED DESCRIPTION OF EMBODIMENTS

As shown in FIGS. 3-4, an infusion needle according to an embodiment of the disclosure is shown, including a needle holder 510 and a needle bar 520. The needle holder 510 is preferably made of a thermoplastic material adapted for injection moulding, and especially, a transparent thermoplastic plastic material, such as polycarbonate or polypropylene. The needle bar is preferably made of a stainless steel material.

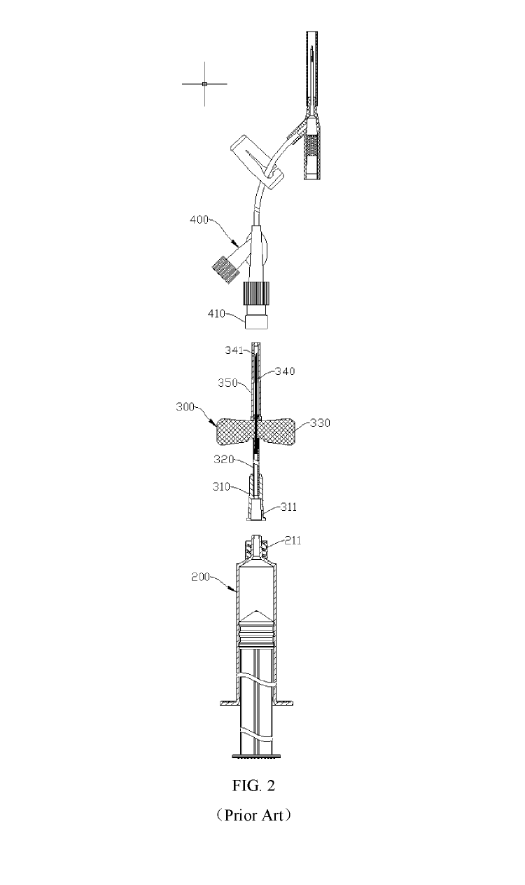

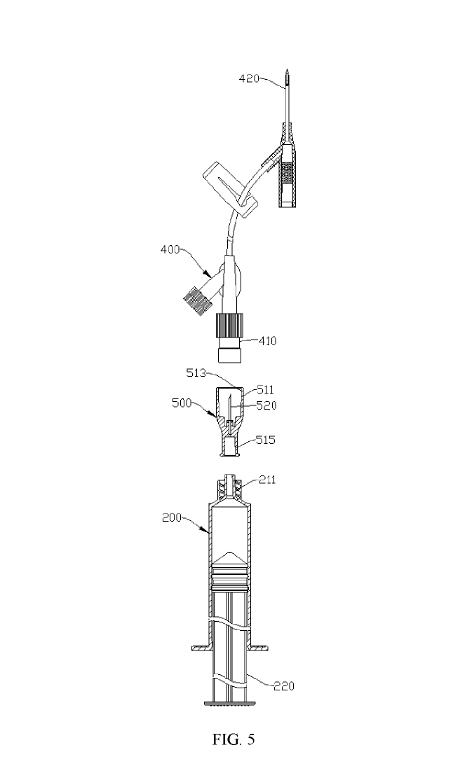

As shown in figures, the needle holder 510 may further include an opening thick end 511 adapted to accommodate at least one part of the heparin cap 410 shown in FIG. 2, and an opening thin end 515 adapted to be coupled to and in fluid communication with the upper part of the medicinal fluid supply device 200 shown in FIG. 2 (the more specific cooperative relationship is shown schematically in FIG. 5). Before the whole description of the connection and cooperation between the needle holder and the needle bar, a preferred embodiment of the opening thick end 511 and the opening thin end 515 will be described in detail in combination with FIGS. 3-4.

(1) The opening thick end 511 has a substantially cylinder-shaped side wall 512 and a substantially cylinder-shaped inner wall 513, the side wall 512 preferably has a shape of a cylinder or a regular polygon cylinder with at least six sides, and the inner wall 513 preferably has a shape of a cylinder or a conical cylinder. The diameter of the opening of the inner wall is denoted by A, and A is preferably larger than 8.5 mm.

(2) The opening thin end 515 includes an inner wall 516 that follows ISO 594-2:1998 and GB/T 1962.2-2001 standards, and a side wall 517 with a lug 518. The opening thin end 515 is a standard 6% (Luer) inner tapered locking joint that follows the medical industry standards, adapted to be engaged with the outer tapered locking joint 211 of the medicinal fluid supply device such as syringe 200.

It can be understood that the above specific limitations to the opening thick end 511 and the opening thin end 515 are exemplary embodiments. It should be noted that variations and improvements will become apparent to those skilled in the art, including providing the opening thick end and the opening thin end with different shapes, sizes and relative position relationships, according to practical requirements and different components related to the opening thick end and the opening thin end (for example, different shapes and sizes of the heparin cap, and different standard joints of the syringe), without departing from its spirit and scope.

The opening thick end is generally used to accommodate and guide the heparin cap 410, and the opening thin end is generally used to be coupled to/in communication with the medicinal fluid supply device, so sometimes the opening thick end is also called the “guiding unit” in context, and the opening thin end is also called the “connecting unit”. It should be noted that the above different names name a same component according to the shape and the function respectively, so there is no difference between the opening thick end and the guiding unit and between the opening thin end and the connecting unit in the substantial sense.

Back to FIGS. 3-4, the opening thick end 511 and the opening thin end 515 are combined together at the central position of the needle holder 510, and have a common bottom layer 531. The central position of the bottom layer is provided with a boss 541 extending along the opening direction of the thick end, and a ladder-shaped cylindrical bore 534 passing through the bottom layer 531 and the boss 541 making the opening thick end in communication with the opening thin end. Preferably, the ladder-shaped cylindrical bore 534 is coaxial with the inner wall 513 of the opening thick end 511. At least one shoulder 551 is arranged between the bottom layer 531, the boss 541 and the inner wall 513. The top of the shoulder is provided with a plane 552 which is parallel to the end plane 519 of the opening thick end, the diameter of the circumference where the plane 552 is combined with the inner wall 513 is denoted by A, and the distance between the plane 552 and the end plane 519 of the opening thick end is denoted by C. B is preferably larger than 8 mm, and C is preferably less than 12 mm.

The needle bar 520 includes a proximal end 521, a distal end 523 and a lumen 522 passing through the needle bar 520. The proximal end 521 is preferably fixed in the ladder-shaped cylindrical bore 534 by adhesion. The distal end 523 may include a blunt tip end or a pointed tip end 524 shown in FIG. 4. The distance between the thick portion of the tip end 524 and the plane of the top of the shoulder 551 is denoted by D, and the distance between the tip point of the tip end 524 and the end plane 519 of the opening thick end is denoted by E. D is preferably larger than 5 mm, and E is preferably larger than 2 mm. Communication between the opening thick end and the opening thin end can be established through the lumen 522 of the needle bar 520. In a preferred embodiment, the needle bar 520 is coaxial with the inner wall 513 of the thick end 511.

The needle bar 520 is generally used to puncture the heparin cap, so sometimes the needle bar 520 is also called the “puncturing unit” in context. Similar to the above explanation, there is no difference between the names of the needle bar and the puncturing unit in the substantial sense.

The following will describe how to use the infusion needle 500 according to an embodiment of the disclosure, in combination with the heparin cap and the medicinal fluid supply device.

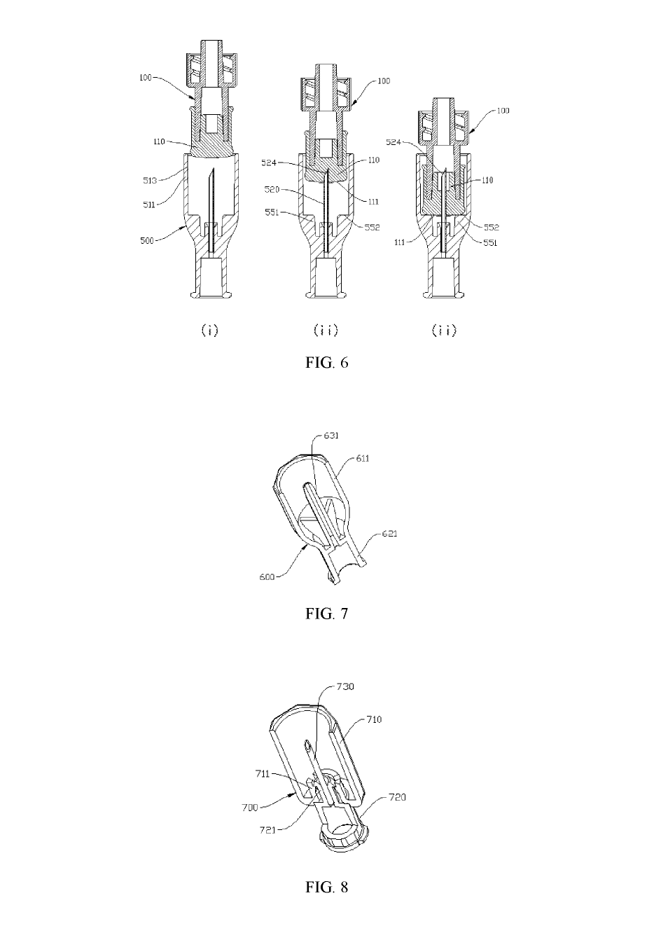

As shown in FIG. 5, the opening thin end 515, the opening thick end 511 and the needle bar 520 of the heparin cap puncture infusion needle 500 perform the following actions respectively to achieve the technological purpose of the disclosure. The opening thick end 511 is connected to a corresponding joint (for example, a 6% (Luer) outer tapered locking joint 221 of the syringe 200 shown in FIG. 5) of the medicinal fluid supply device with a known method; the inner wall 513 of the opening thick end 511 cover (i.e., accommodate) the heparin cap of the vascular access device or the three-way valve device (for example, the heparin cap 410 used for the indwelling needle 400); and the needle bar can penetrate (guided by the inner wall 513) and enter inside the heparin cap 410 in the opening thick end, and FIGS. 6(i)-(iii) sequentially shows the process of the needle bar penetrating into the heparin cap.

As shown in FIG. 6, according to a preferred embodiment of the disclosure, the diameter of the inner wall 513 of the opening thick end 511 of the heparin cap puncture infusion needle 500 is slightly larger than the outer diameter of the sealing rubber end 110 of the heparin cap 100. The process of the needle bar penetrating into the heparin cap may be generally divided into the following three stages:

(1) Firstly, when the sealing rubber end 110 of the heparin cap 100 is inserted into the heparin cap puncture infusion needle 500 along the inner wall 513, the needle bar 520 and the sealing rubber end 110 approach each other (see FIG. 6(i)). The needle body is always located within the opening thick end 511 when the needle body approaches the sealing rubber end, so the risk of the medical worker or patient being punctured is greatly reduced.

(2) Then, the tip end 524 of the needle bar touches the sealing rubber, and the tip end 524 is substantially perpendicularly inserted within the heparin cap from the central position of the sealing rubber 100 of the heparin cap (see FIG. 6(ii)). As explained in the above background, if the position and/or direction in which the needle bar penetrates are improper, there may be some unfavorable consequences, such as plastic scraps production, or the tip being buried under the plastic body. The disclosure has effectively solved the above problem through providing an appropriate position and direction of the tip end 524 relative to the sealing rubber end 110. In addition, it should be understood by those skilled in the art that the needle bar 520 may penetrate into the sealing rubber end 110 in other positions or other directions relative to the sealing rubber end 110 according to special needs, through appropriately arranging the relative positioning between the opening thick end 511 and the needle bar 520.

(3) Finally, when the end surface 111 of the heparin cap touches the plane 552 of the top of the shoulder 551 at the bottom of the inner wall 513, and cannot move on (that is, the position of the heparin cap is limited), the puncture tip end 524 of the needle bar stays inside the heparin cap 100 near the sealing rubber end 110 (see FIG. 6(iii)). As explained in the above background, such positioning method facilitates the medical worker to completely wash the medicinal liquid remained within the heparin cap off during washing, to effectively avoid medical accident caused by the incompatibility between the medicinal liquid to be infused and the medicinal liquid remained.

The system assembled according to the above process may be referred to as vascular access system, in which the opening thin end 515 of the heparin cap puncture infusion needle 500 can be connected to a joint of the syringe 200, and the needle bar 520 can penetrate into the heparin cap 410 of the indwelling needle 400. The vascular access system according to the disclosure ensures an appropriate fluid passageway to be created between the syringe 200 and the human blood vessel (not shown) connected to the needle bar 420 of the indwelling needle, and the medicinal liquid in the syringe can be infused into the human blood vessel by the medical worker pushing the push rod 220 of the syringe, to achieve the corresponding medical purposes.

FIG. 7 shows a heparin cap puncture infusion needle 600 according to another embodiment of the disclosure. In the embodiment, the components of the heparin cap puncture infusion needle 600 are integrally formed. The integral structure can be subdivided into an opening thick end portion 611, an opening thin end portion 621 and a needle bar portion 631, and the three portions can be made of a same plastic material, and preferably made by one-shot plastic forming with injection moulding. The integrally formed infusion needle 600 can further reduce manufacturing cost and labor cost, and can be directly used without user installation.

FIG. 8 shows a heparin cap puncture infusion needle 700 according to a further embodiment of the disclosure. In the embodiment, the heparin cap puncture infusion needle 700 can be divided into three separate components: an opening thick end component 710, an opening thin end component 720 and a needle bar component 730. The opening thick end component 710 and the opening thin end component 720 are preferably made of a thermoplastic material adapted for injection moulding, and especially, a transparent thermoplastic plastic material, such as polycarbonate or polypropylene. The needle bar component 730 is preferably made of a stainless steel material. As shown in FIG. 8, the needle bar component 730 and the opening thin end component 720 are fixed together by adhesion, and the opening thick end component 710 and the opening thin end component 720 are connected together by physical means (such as snap fittings 711, 721 in the embodiment). Preferably, in the embodiment of FIG. 8, the opening thick end component 710 and the opening thin end component 720 are connected together by a mechanical means (for example, a snap fitting with a barb) to form an assembly which is non-removable or is very difficult to disassemble, to ensure the single-use of the medical device, and ensure the medical safety of the whole process of medicinal liquid supply.

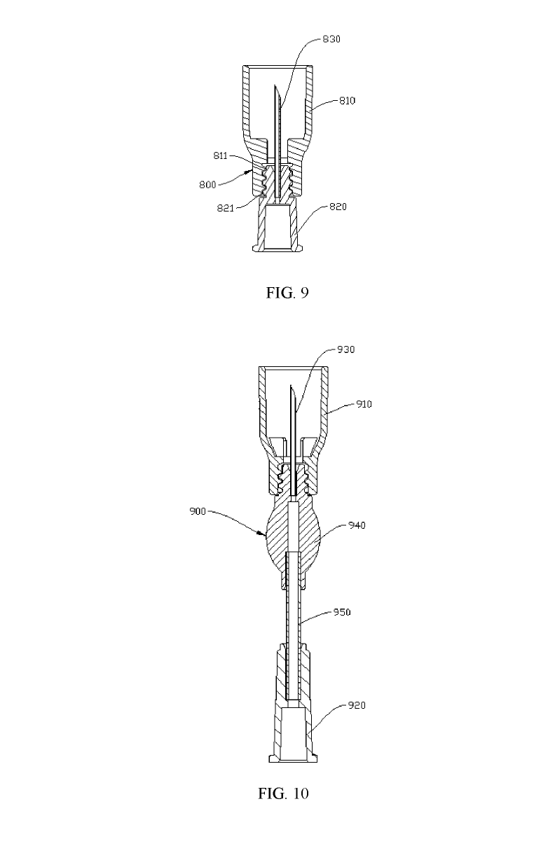

FIG. 9 shows a heparin cap puncture infusion needle 800 according to a further embodiment of the disclosure. In the embodiment, the heparin cap puncture infusion needle 800 is still divided into three separate components: an opening thick end component 810, an opening thin end component 820 and a needle bar component 830. The difference from the embodiment of FIG. 8 is that the opening thick end component 810 and the opening thin end component 820 are not directly mechanically connected with each other, but stringed together through the needle bar component 830. In addition, as shown in FIG. 10, the opposite sides of the opening thick end component 810 and the opening thin end component 820 are provided with positioning components 811 and 821, to assist the connection between the opening thick end component 810 and the opening thin end component 820. Preferably, the opening thick end component 810, the opening thin end component 820 and the needle bar component 830 in FIG. 9 are removable from each other. FIG. 10 shows a heparin cap puncture infusion needle 800 according to a further embodiment of the disclosure. In the embodiment, the heparin cap puncture infusion needle 900 is divided into five separate components: an opening thick end component 910, an inner tapered joint component 920, a needle bar component 930, a needle holder component 940, and a catheter component 950. The inner tapered joint component 920 is fixed to the catheter component 950 by adhesion, the catheter component 950 is fixed to the needle holder component 940 by adhesion, the needle holder component 940 is fixed to the needle bar 930 by adhesion, and the needle holder component 940 is detachedly connected to the opening thick end component 910 by thread. The difference from the embodiments of FIG. 8 and FIG. 9 is that the opening thick end component 910 and the opening thin end component 920 are physically separate, and are fluid coupled together only through the needle bar component 930, the catheter component 950 and the needle holder component 940. In other words, the medicinal liquid from the opening thin end component 920 (for example, syringe) can reach the opening thick end component 910 through the fluid passageway (the catheter component 950, the needle holder component 940 and the needle bar component 930) as shown in FIG. 10, to endure the normal transmission of the medical fluid when needed.

Although some middle parts may be added as separating the opening thick end component 910 and the opening thin end component 920, to improve cost, the separable heparin cap puncture infusion needle 900 undoubtedly increases the flexibility to ensure the therapeutic process to be effective.

Although there is not specifically shown, it should be understood that the heparin cap puncture infusion needle 600, 700, 800 and 900 can be connected to the syringe 200 and the heparin cap 410 of the indwelling needle 400 respectively, similar to the means described above in combination with FIGS. 5-6, and create a fluid passageway between the syringe and the patient blood vessel through a puncture infusion needle penetrating into the heparin cap.

Although various alternative embodiment of the disclosure are exemplarily provided above, it is easy for those skilled in the art to think of other variations, replacements or alterations by reading the above disclosure. For example, the relative positions of the connecting unit, the guiding unit and the puncturing unit may be different from the above embodiments (for example, the connecting unit, the guiding unit and the puncturing unit are not located and are not substantially coaxial), which still falls into the scope of the disclosure. Also for example, in order to reduce the overall size of the device for puncturing heparin cap, the puncturing unit is not wholly but partially located in the guiding unit. Also for example, two components of the connecting unit, the guiding unit and the puncturing unit can be made into a large component as a whole, and the other component may be in removable or non-removable connection and/or fluid communication with the large component, or the other component may be only in fluid communication with (i.e., physically separate from) the large component.

In addition, when there is no conflict, the limitations in various embodiments can be combined with each other. For example, it is entirely possible to partially or wholly apply the material or manufacturing mode of some component mentioned in a certain embodiment to the component in other embodiments, which is easy to think out, so it does not go beyond the teaching and scope of the disclosure.

In conclusion, the exemplary embodiments described above disclose the preferred embodiments of the disclosure, and make those skilled in the art be able to practice the disclosure, including the method for making and using any device or system and executing any combination. However, the scope of the present disclosure is defined by the appended claims, which may include other embodiments that those skilled in the art can think out.

1. A device for puncturing a heparin cap comprising:
a connecting unit adapted to be coupled to a medicinal fluid supply device in fluid communication;
a guiding unit adapted to accommodate at least one part of the heparin cap; and
a puncturing unit for puncturing the heparin cap, configured to be in fluid communication with the connecting unit and at least partially located on a predetermined position in the guiding unit,
wherein the predetermined position is configured so that in the process of at least one part of the heparin cap entering into the guiding unit, the puncturing unit is inserted into a sealing rubber of the heparin cap in an appropriate location in the heparin cap at an appropriate angle relative to the heparin cap, and ultimately penetrate through the sealing rubber.

2. The device of claim 1, wherein the appropriate angle comprises that the puncturing unit is perpendicular to the sealing rubber, and the appropriate location comprises a central portion of the sealing rubber.

3. The device of claim 1, wherein the guiding device further comprises a position-limiting component, configured to limit a final depth of the heparin cap entering into the guiding unit.

4. The device of claim 3, wherein the position-limiting component comprises at least one of a bulge arranged within the guiding unit, and a tapered shape of the guiding unit in a direction from the heparin cap to the guiding unit.

5. The device of claim 1, wherein at least two of the connecting unit, the guiding unit and the puncturing unit are integrally formed.

6. The device of claim 5, wherein the connecting unit, the guiding unit and the puncturing unit are integrally formed.

7. The device of claim 1, wherein at least two of the connecting unit, the guiding unit and the puncturing unit are separately formed and detachably assembled.

8. The device of claim 7, wherein the connecting unit, the guiding unit and the puncturing unit are separately formed, the outer surface of the puncturing unit is adhered onto the inner surface of the connecting unit, and the connecting unit is fitted into the guiding unit.

9. A vascular access system, comprising:
a vascular access device;
a heparin cap coupled to the vascular access device;
a device for puncturing heparin cap according to claim 1; and
a medicinal fluid supply device in fluid communication with the device for supplying medicinal fluid.

10. The vascular access system of claim 9, where in the vascular access device comprises at least one of an indwelling needle, a central venous catheter, a peripherally inserted central catheter and an artery catheter.

11. The device of claim 2, wherein the guiding device further comprises a position-limiting component, configured to limit a final depth of the heparin cap entering into the guiding unit.

12. The device of claim 11, wherein the position-limiting component comprises at least one of a bulge arranged within the guiding unit, and a tapered shape of the guiding unit in a direction from the heparin cap to the guiding unit.

13. The device of claim 2, wherein at least two of the connecting unit, the guiding unit and the puncturing unit are integrally formed.

14. The device of claim 13, wherein the connecting unit, the guiding unit and the puncturing unit are integrally formed.

15. The device of claim 2, wherein at least two of the connecting unit, the guiding unit and the puncturing unit are separately formed and detachably assembled.

16. The device of claim 15, wherein the connecting unit, the guiding unit and the puncturing unit are separately formed, the outer surface of the puncturing unit is adhered onto the inner surface of the connecting unit, and the connecting unit is fitted into the guiding unit.

17. A vascular access system, comprising:
a vascular access device;
a heparin cap coupled to the vascular access device;
a device for puncturing heparin cap according to claim 2; and
a medicinal fluid supply device in fluid communication with the device for supplying medicinal fluid.

18. The vascular access system of claim 17, where in the vascular access device comprises at least one of an indwelling needle, a central venous catheter, a peripherally inserted central catheter and an artery catheter.
